(19) United States
(12) Patent Application Publication
(10) Pub. No.: US 2026/0204425 A1
(43) Pub. Date: Jul. 16, 2026

(54) System and Method for Chat-Based Training Simulations for Mental Health Professionals Using Large Language Models and Evidence-Based Psychological Principles

(71) Applicant: Ryan Michael Torrie, Westminster, CA (US)

(72) Inventor: Ryan Michael Torrie, Westminster, CA (US)

(21) Appl. No.: 19/017,753

(22) Filed: Jan. 12, 2025

(51) Int. Cl. G16H 50/50; G16H 20/70; G16H 40/20

(52) U.S. Cl. CPC G16H 50/50; G16H 20/70; G16H 40/20

(57) ABSTRACT

The present invention provides a system and method for delivering interactive, chat-based training simulations tailored to mental health professionals. The system leverages large language model (LLM) technology, integrated with evidence-based psychological theories and behavioral principles, to create realistic simulations of interactions with virtual patients or colleagues. Key components include a prompting engine for generating scenario-specific prompts, an LLM for producing contextually accurate and clinically consistent responses, a transcript management module for recording and maintaining conversation logs, and an analytical feedback engine for evaluating user performance and providing actionable insights. The system enables mental health practitioners to practice therapeutic techniques, enhance clinical understanding, and refine professional skills in a controlled, immersive, and cost-effective environment. Applications extend to organizational training, candidate assessments, and broader professional development scenarios, offering an innovative, scalable solution to enhance the quality and accessibility of mental health education and training.

### BACKGROUND OF THE INVENTION

### Field of Invention

The present invention relates to the field of training and simulation technologies, particularly systems and methods for interactive education and skills development. Specifically, it pertains to the use of large language model (LLM) technology integrated with evidence-based psychological theories and principles to create AI-driven, chat-based simulations. These simulations are designed for training mental health professionals, including licensed professional counselors, psychologists, and related practitioners, with applications extending to organizational training and candidate assessment in the field of mental healthcare and beyond.

### BRIEF SUMMARY OF THE INVENTION

The present invention provides a system and method for delivering interactive, AI-driven training simulations tailored to mental health professionals. Leveraging large language model (LLM) technology, the system incorporates evidence-based psychological theories, behavioral expectations, and scenario narratives to simulate realistic interactions with virtual patients or colleagues. These simulations allow practitioners to practice therapeutic techniques, improve their understanding of psychological phenomena, and develop professional skills in a controlled and immersive environment. The invention enhances the quality of mental health training by offering accessible, on-demand simulations that replicate clinical scenarios with high fidelity, addressing the limitations of traditional role-playing and non-interactive online courses.

### BRIEF DESCRIPTION OF THE FIGURES

FIG. 1: This FIGURE illustrates the operational workflow of the simulation system, beginning with the definition of training objectives and scenarios, progressing through prompt generation, simulation initialization, real-time user interaction, transcript management, data analysis, and culminating in the generation of a detailed feedback report to enhance user skills and performance.

### DETAILED DESCRIPTION

The present invention provides a comprehensive system and method for delivering interactive, AI-driven training simulations specifically designed for mental health professionals. This invention integrates large language model (LLM) technology with established psychological theories, behavioral research, and clinical practices to create a scalable and immersive platform for skill development, professional training, and educational assessments. By addressing the limitations of existing training modalities, the invention enhances the efficacy, accessibility, and realism of mental health education. The system is composed of several core components that work together to create a seamless simulation experience.

At the core of the system is a prompt generation module, which encodes training objectives into structured narrative frameworks. These prompts incorporate psychological principles, therapeutic techniques, and behavioral guidelines to define scenarios such as patient profiles, clinical conditions, and therapeutic challenges. The structured prompts are sent to a pre-trained LLM, which has been fine-tuned with domain-specific datasets to enhance its understanding of psychological phenomena and clinical practices. The LLM generates responses that emulate realistic psychological states and clinical scenarios, ensuring that the interactions are contextually accurate and aligned with the training objectives.

A transcript management system dynamically records interactions between the user and the virtual patient or colleague, ensuring continuity and providing the LLM with real-time contextual data for coherent responses. This transcript also serves as a source for post-simulation analysis. The analytical feedback engine processes these transcripts using natural language processing (NLP) algorithms, and optionally, a secondary LLM. This engine identifies patterns in user behavior, assesses the application of therapeutic techniques, and provides actionable feedback for performance improvement. Anomalies or edge cases flagged by the engine may undergo manual review for further refinement of the system.

The system is administered through a user-friendly interface that allows trainers and administrators to define learning objectives, select or customize scenarios, and review post-simulation analytics. This interface supports modular expansion and integration with external learning management systems (LMS) or organizational training platforms. The operational workflow begins with administrators defining simulation objectives, followed by initializing the scenario through the prompt generation module. Users then engage in real-time interactions with the virtual entities, which are driven by the LLM's text-based responses. Upon completion, the system generates a detailed feedback report for the user, highlighting strengths, areas for improvement, and recommendations for further practice.

The invention offers distinct advantages over traditional training methods. By leveraging advanced LLM technology, it provides high-fidelity simulations that emulate complex psychological phenomena, allowing users to practice therapeutic techniques in a risk-free, interactive environment. Unlike role-playing exercises, which are often limited by human variability and logistical challenges, this system delivers consistent, repeatable, and accessible scenarios. Additionally, the analytical feedback engine ensures a data-driven approach to learning by offering objective insights into user performance.

This system is designed primarily for mental health professionals, including counselors, psychologists, and social workers, as well as organizations that employ or train such professionals. However, its applications extend beyond mental health, with potential use cases in fields such as education, law enforcement, and human resources, where simulation-based training can enhance decision-making and professional development. The invention's modular design and scalable architecture enable its adaptability to a wide range of industries and training needs, ensuring its relevance in various professional contexts.

Below is a Python implementation of the described 7-step flow. This code demonstrates the core logic of the system, including defining objectives, generating prompts, initializing the simulation, handling interactions, logging transcripts, analyzing data, and generating feedback.

```
  import random
  import json
  from datetime import datetime
  class TrainingSimulation:
    def __init__(self, objectives, scenario_details):
      self.objectives = objectives
      self.scenario_details = scenario_details
      self.transcript = [ ]
      self.feedback = { }
    def define_objectives(self):
      print(f“Defining Objectives: {self.objectives}”)
      return f“Training Objectives: {self.objectives}”
    def generate_prompts(self):
      prompts = {
        “patient_profile”: self.scenario_details.get(“patient_profile”, “Generic
patient”),
        “clinical_challenge”: self.scenario_details.get(“clinical_challenge”,
“General challenge”),
        “desired_outcome”: self.scenario_details.get(“desired_outcome”, “General
skill development”)
      }
      print(f“Generated Prompts: {prompts}”)
      return prompts
    def initialize_simulation(self, prompts):
      print(“Initializing Simulation...”)
      initial_message = f“Welcome to the simulation. Meet your patient:
{prompts[‘patient_profile’]} facing {prompts[‘clinical_challenge’]}.”
      self.transcript.append({“system”: initial_message})
      return initial_message
    def interact(self, user_input):
      print(f“User Input: {user_input}”)
      # Simulated response logic
      response = f“The virtual patient responds:
“{self.generate_response(user_input)}”
      self.transcript.append({“user”: user_input, “system”: response})
      return response
    def generate_response(self, user_input):
      # Mock LLM response logic
      simulated_responses = [
        “Can you explain that a bit more?”,
        “I feel anxious about that.”,
        “What do you mean by that?”,
        “I need some help with my thoughts.”
      ]
      return random.choice(simulated_responses)
    def log_transcript(self):
      print(“Logging Transcript...”)
      with
open(f“transcript_{datetime.now( ).strftime(‘%Y%m%d_%H%M%S’)}.json”, “w”) as f:
        json.dump(self.transcript, f, indent=4)
      print(“Transcript saved.”)
      return self.transcript
    def analyze_data(self):
      print(“Analyzing Data...”)
      # Mock analysis
      insights = {
        “strengths”: “Good use of open-ended questions.”,
        “weaknesses”: “Missed opportunities to explore patient emotions.”,
        “recommendations”: “Practice reflective listening and empathy-based
responses.”
      }
      self.feedback = insights
      return insights
    def generate_feedback(self):
      print(“Generating Feedback...”)
      feedback_report = {
        “Summary”: “Simulation completed successfully.”,
        “Insights”: self.feedback,
        “Transcript”: self.transcript
      }
      with
open(f“feedback_{datetime.now( ).strftime(‘%Y%m%d_%H%M%S’)}.json”, “w”) as f:
        json.dump(feedback_report, f, indent=4)
      print(“Feedback saved.”)
      return feedback_report
  # Example Usage
  if __name__ == “__main__”:
    # Step 1: Define Objectives and Scenarios
    objectives = “Practice reflective listening and cognitive-behavioral techniques.”
    scenario_details = {
      “patient_profile”: “John Doe, a 35-year-old experiencing anxiety”,
      “clinical_challenge”: “Difficulty managing work-related stress”,
      “desired_outcome”: “Learn how to apply CBT methods to reduce anxiety.”
    }
    # Create Training Simulation instance
    simulation = TrainingSimulation(objectives, scenario_details)
    # Step 2: Generate Prompts
    prompts = simulation.generate_prompts( )
    # Step 3: Initialize Simulation
    print(simulation.initialize_simulation(prompts))
    # Step 4: Interact with Virtual Patient
    print(simulation.interact(“How can I help you today?”))
    print(simulation.interact(“Tell me more about your work stress.”))
    # Step 5: Log Simulation Transcript
    simulation.log_transcript( )
    # Step 6: Analyze Simulation Data
    insights = simulation.analyze_data( )
    print(f“Insights: {insights}”)
    # Step 7: Generate Post-Simulation Feedback
    feedback_report = simulation.generate_feedback( )
    print(f“Feedback Report: {feedback_report}”)
```

### Detailed Description of Figures

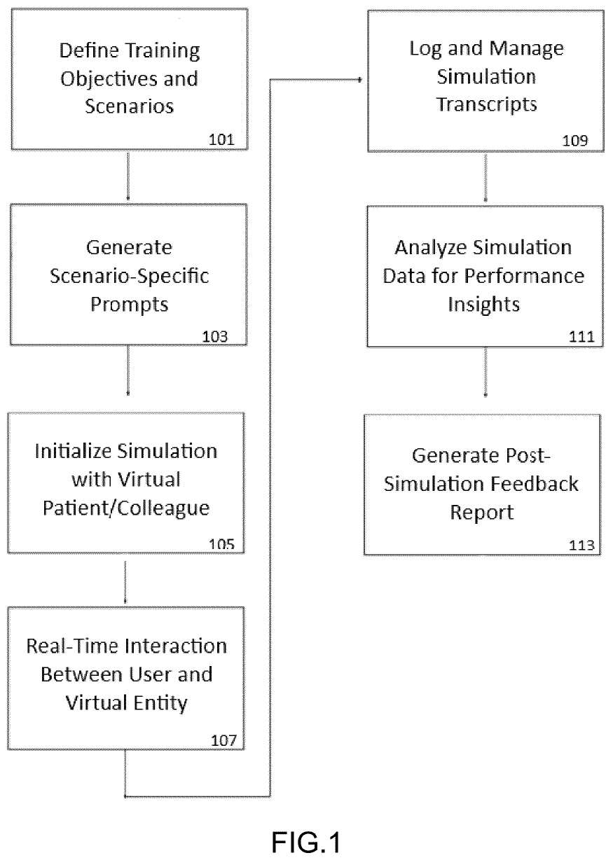

FIG. 1.101: Define Training Objectives and Scenarios—This step involves trainers or administrators setting specific goals for the simulation. Objectives may include practicing therapeutic techniques, understanding psychological phenomena, or addressing clinical scenarios. The training administrator defines the context, such as patient demographics, presenting problems, and desired learning outcomes. These parameters form the foundation for the scenario to ensure alignment with educational objectives.

FIG. 1.103: Generate Scenario-Specific Prompts—The system's prompting engine encodes the defined objectives into structured prompts. These prompts specify the behavioral expectations, narrative elements, and psychological principles relevant to the scenario. The prompts are designed to guide the simulation's flow and ensure the large language model (LLM) generates responses that align with clinical and educational goals.

FIG. 1.105: Initialize Simulation with Virtual Patient/Colleague—The structured prompts are sent to the LLM, which generates an introductory narrative to establish the simulation context. The virtual patient or colleague is introduced, complete with realistic psychological and behavioral attributes. This initialization ensures that the user is provided with a clear and immersive starting point for the interaction.

FIG. 1.107: Real-Time Interaction Between User and Virtual Entity—The user engages in a simulated dialogue with the virtual patient or colleague through a chat-based interface. The LLM dynamically generates responses based on the user's input and the simulation's context. This step allows users to practice therapeutic techniques and decision-making in real-time while receiving realistic feedback from the virtual entity.

FIG. 1.109: Log and Manage Simulation Transcripts—During the simulation, all interactions are logged by the transcript management system. This module records dialogue, tracks user actions, and provides context for subsequent responses generated by the LLM. The transcript ensures continuity during the simulation and serves as a critical data source for post-simulation analysis.

FIG. 1.111: Analyze Simulation Data for Performance Insights—Upon completion of the simulation, the analytical feedback engine processes the transcript. The engine evaluates user performance by comparing applied techniques and decision-making processes against predefined benchmarks. Key patterns, edge cases, and areas for improvement are identified through natural language processing (NLP) and machine learning techniques.

FIG. 1.113: Generate Post-Simulation Feedback Report—The system generates a comprehensive feedback report for the user. This report summarizes their strengths, identifies weaknesses, and provides actionable recommendations for further training. It also highlights specific moments in the simulation where the user performed well or deviated from best practices, offering a roadmap for skill refinement.

In general, machine learning algorithms are used to make a prediction or classification regarding user performance by comparing applied techniques and decision-making processes against predefined benchmarks. Based on some input data, which can be labeled or unlabeled, the algorithm will produce an estimate about a pattern in the data.

An error function evaluates the prediction of the model. If there are known examples, an error function can make a comparison to assess the accuracy of the model. A model optimization process then occurs. If the model can fit better to the data points in the training set, then weights are adjusted to reduce the discrepancy between the known example and the model estimate. The algorithm will repeat this “evaluate and optimize” process, updating weights autonomously until a threshold of accuracy has been met.

Supervised learning in particular uses a training set to teach models to yield the desired output. This training dataset includes inputs and correct outputs, which enables the model to learn over time. The algorithm measures its accuracy through the loss function, adjusting until the error has been sufficiently minimized. Thus, through the computer-implemented process described above, the present invention can improve its ability to predict and detect e.g., user performance by comparing applied techniques and decision-making processes against predefined benchmarks.

After training, the machine learning categorization engine processes the sensor data using pre-trained models trained on datasets of users'performance. It comprises an application-specific integrated circuit (ASIC) for an artificial neural network connected to the computer memory device, the ASIC comprising: a plurality of neurons organized in an array, wherein each neuron comprises a register, a processing element and at least one input, and a plurality of synaptic circuits, each synaptic circuit including a memory for storing a synaptic weight, wherein each neuron is connected to at least one other neuron via one of the plurality of synaptic circuits, wherein the array is configured to analyze said user performance by comparing applied techniques and decision-making processes against predefined benchmarks trained on historical datasets associating other users'performance on predefined benchmarks, wherein the AI/ML categorization engine makes a prediction regarding the performance of the current user.

What is claimed is:

1. A system for delivering interactive, chat-based training simulations for mental health professionals, comprising:
a. a prompting engine configured to generate scenario-specific prompts based on psychological principles, behavioral expectations, and narrative structures;
b. a large language model (LLM) integrated with the system, wherein the LLM is fine-tuned to generate text-based responses that emulate psychological and clinical states of virtual patients or colleagues;
c. a transcript management module configured to dynamically record interactions during the simulation and provide context for subsequent responses;
d. an analytical feedback engine configured to process the recorded transcripts, evaluate user performance, and provide actionable insights for performance improvement, comprising an application-specific integrated circuit (ASIC) for an artificial neural network connected to the computer memory device, the ASIC comprising: a plurality of neurons organized in an array, wherein each neuron comprises a register, a processing element and at least one input, and a plurality of synaptic circuits, each synaptic circuit including a memory for storing a synaptic weight, wherein each neuron is connected to at least one other neuron via one of the plurality of synaptic circuits, wherein the array is configured to analyze said user performance by comparing applied techniques and decision-making processes against predefined benchmarks trained on historical datasets associating other users'performance on predefined benchmarks, wherein the AI/ML categorization engine makes a prediction regarding the performance of the current user; and
e. a user interface enabling configuration of training scenarios, real-time interaction with virtual entities, and review of post-simulation analytics.

2. The system of claim 1, wherein the prompting engine is further configured to:
a. encode training objectives into structured prompts that define psychological phenomena, clinical conditions, and desired user behaviors;
b. adapt prompts based on user inputs during the simulation to ensure alignment with training goals.

3. The system of claim 1, wherein the large language model (LLM) is trained on domain-specific datasets to ensure responses are:
a. contextually relevant;
b. clinically accurate; and
c. aligned with established therapeutic practices.

4. The system of claim 1, wherein the transcript management module is further configured to:
a. provide real-time contextual data from ongoing simulations to the LLM to ensure coherence in responses;
b. store transcripts for post-simulation analysis and iterative improvement of the system.

5. The system of claim 1, wherein the analytical feedback engine is further configured to:
a. utilize natural language processing (NLP) algorithms to identify patterns in user interactions;
b. assess the appropriateness and effectiveness of therapeutic techniques applied by the user;
c. recommend areas for improvement based on predefined benchmarks or training objectives.

6. The system of claim 1, wherein the user interface is further configured to:
a. allow trainers to customize scenarios, define learning objectives, and select specific psychological phenomena for simulation;
b. present users with detailed post-simulation feedback reports, including strengths, weaknesses, and actionable recommendations.

7. A method for delivering interactive, chat-based training simulations for mental health professionals, comprising:
a. generating a training scenario using a prompting engine that encodes psychological principles and therapeutic objectives into a structured prompt;
b. initializing a simulation by providing the structured prompt to a large language model (LLM);
c. engaging a user in real-time interaction with a virtual patient or colleague, wherein the LLM generates text-based responses based on the prompt and user inputs;
d. recording interactions dynamically in a transcript management module to provide context for ongoing interactions and enable post-simulation analysis;
e. analyzing the recorded transcript using an analytical feedback engine to evaluate user performance and identify areas for improvement; and
f. providing the user with a detailed feedback report summarizing performance metrics and recommendations.

8. The method of claim 7, further comprising:
a. refining the large language model's performance over time by analyzing anonymized transcripts from multiple simulations to identify patterns, edge cases, and areas for model improvement.

9. The method of claim 7, wherein the feedback report includes:
a. an assessment of therapeutic techniques applied by the user;
b. identification of behaviors and strategies that align or deviate from clinical best practices;
c. recommendations for further training or development.

10. The method of claim 7, wherein the training scenario can be adapted dynamically during the simulation based on user inputs to address unanticipated user actions or deviations from the intended training objectives.

11. A non-transitory computer-readable medium storing instructions that, when executed by a processor, cause a system to perform the method of claim 7.

12. The system of claim 1, wherein the large language model is augmented with data derived from prior simulations to improve response accuracy and align future interactions with updated psychological research and clinical guidelines.

13. The system of claim 1, wherein the training simulations are accessible via an online platform, enabling users to engage in on-demand training sessions from remote locations.

14. The system of claim 1, further comprising a data privacy and security module configured to:
a. anonymize all stored transcripts to protect user and participant confidentiality;
b. comply with relevant data protection regulations, including but not limited to HIPAA and GDPR.

15. The system of claim 1, wherein the simulations can be extended for applications beyond mental health training, including but not limited to:
a. candidate assessments during organizational hiring processes;
b. skills development in other professional fields, such as law enforcement, education, and human resources.
